SEQUENCE LISTING (5 sequences)

SEQ ID NO: 1 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - protein, 30 aa, synthetic construct
  REGION 1..30: note = peptide linker
GGGGSGGGGSGGGGSGGGGSGGGGSGGGGS

SEQ ID NO: 3 - protein, 221 aa, synthetic construct
  REGION 1..221: note = Fc varinat
PPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 680 aa, synthetic construct
  REGION 1..680: note = alpha galactosidase-Fc fusion protein
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLLGGGGSGGGGSGGGGSGGGGSGGGGSGGGGSPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - DNA, 2043 bp, synthetic construct
  misc_feature 1..2043: note = alpha galactosidase-Fc fusion protein
  source 1..2043: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttacttggcggcggaggttcaggtggtggtggctctggcggtggagggtcggggggaggcggctctggaggagggggctccggtgggggaggtagcccaccatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttccaaagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgcatgaggctctgcacaaccactacacgcagaagagcctctccctgtctctgggtaaatga

(19) United States
(12) Patent Application Publication
(10) Pub. No.: US 2025/0099558 A1
(43) Pub. Date: Mar. 27, 2025

(54) LYOPHILIZED FORMULATION COMPRISING A FUSION PROTEIN INCLUDING a-GALACTOSIDASE A

(71) Applicants: GREEN CROSS CORPORATION, GYEONGGI-DO (KR); HANMI PHARM. CO., LTD., GYEONGGI-DO (KR)

(72) Inventors: Miroo KIM, Yongin-si, Gyeonggi-do (KR); Miri YOO, Yongin-si, Gyeonggi-do (KR); Jae Woon SON, Yongin-si, Gyeonggi-do (KR); Da-eui PARK, Yongin-si, Gyeonggi-do (KR); Shin-Ae YI, Yongin-si, Gyeonggi-do (KR); Jin Young KIM, Hwaseong-si, Gyeonggi-do (KR); Jun Sub PARK, Hwaseong-si, Gyeonggi-do (KR); Doo Seo JANG, Hwaseong-si, Gyeonggi-do (KR); Sung Hee HONG, Hwaseong-si, Gyeonggi-do (KR); Sang Yun KIM, Hwaseong-si, Gyeonggi-do (KR)

(21) Appl. No.: 18/832,304

(22) PCT Filed: Mar. 10, 2023
(86) PCT No.: PCT/KR2023/003300

§ 371 Date: Jul. 23, 2024

(30) Foreign Application Priority Data
Mar. 15, 2022 (KR) 10-2022-0032091

(51) Int. Cl. A61K 38/47; A61K 9/16; A61K 9/19

(52) U.S. Cl. CPC A61K 38/47; A61K 9/1617; A61K 9/1623; A61K 9/19; C12Y 302/01022

(57) ABSTRACT

The present invention relates to a lyophilized formulation including a fusion protein of α-galactosidase A and a preparation method thereof, wherein the lyophilized formulation not only has storage stability by including a composition providing structural stability to a fusion protein of α-galactosidase A, but also has excellent stability although the fusion protein is contained at a high concentration.

### TECHNICAL FIELD

The present invention relates to a lyophilized formulation including a fusion protein of α-galactosidase A and a preparation method thereof, and a use thereof.

### BACKGROUND ART

Lysosomes are intracellular organelles involved in degradation of proteins, various lipids such as glycolipids and cholesterol, and carbohydrates and recycling of degraded products as primary constituents of new proteins, membrane components, and other molecules. Diseases associated with functions thereof include lysosomal storage diseases (LSDs).

Lysosomal Storage Diseases (LSDs) are diseases that result from defects in genes encoding enzymes present in lysosomes and degrading glycolipid or polysaccharide waste products, and biological activity of lysosomal enzymes is significantly reduced or absent in cells and tissues of individuals with lysosomal storage diseases. Such deficiency of degradative enzymes causes accumulation of substances in large quantities, thereby causing a problem in the function of cells.

Fabry disease, known as a lysosomal storage disease, is an inherited disorder of glycolipid (glycosphingolipid) metabolism resulting from deficient or insufficient activity of alpha-galactosidase A, which is a hydrolase present in lysosomes. Fabry disease, as a representative disease of α-galactosidase A deficiency, is a disease associated with X chromosome, and typical Fabry patients generally have α-galactosidase A activity of less than 1% compared to that of normal people and have a wide range of symptoms including severe pain in the extremities (acroparesthesia), hypohidrosis, corneal and lenticular changes, skin lesions (angiokeratoma), renal failure, cardiovascular disease, pulmonary failure, nervous system symptoms, and stroke.

Fabry disease causes progressive accumulation of globotriaosylceramide (Gb3) in most tissues of the body. The accumulation of Gb3 is mainly found in vascular endothelia. Such progressive accumulation of Gb3 in vascular endothelia leads to ischemia and infarction in organs such as the kidneys, heart, or brain.

A number of studies have been conducted into enzyme-replacement therapy (ERT) as a representative method for treating lysosomal storage disease (Frances M. Platt et al., J Cell Biol. 2012 Nov. 26; 199(5):723-34). Particularly, since lysosomal storage diseases are caused by genetic defects in particular enzymes, a therapy replacing the deficient enzyme is essential. Enzyme-replacement therapy is a standard therapy in lysosomal storage diseases and may have an effect on alleviating existing symptoms or delaying the progress of the disease by replacing the deficient enzyme. Accordingly, studies have been conducted on various formulations including α-galactosidase A for preventing and treating α-galactosidase A deficiency.

However, in the case of formulations, a composition of a formulation for obtaining stability significantly varies according to a structure, physicochemical properties, dosage, etc., of an active ingredient (e.g., protein drug), and thus it is essential to develop a particular composition suitable for the active ingredient, and development of a formulation including α-galactosidase A is insufficient. Particularly, in the case of a lyophilized formulation including an α-galactosidase A fusion protein at a high concentration, storage stability considerably deteriorates due to a problem such as protein precipitation, and thus conventional lyophilized formulations include α-galactosidase A fusion protein at a concentration of 5 mg/mL or less. However, there is a need to develop a highly stable formulation including a high-concentration α-galactosidase A fusion protein to increase efficacy.

### DISCLOSURE OF INVENTION

### Technical Problem

Development of a stable lyophilized formulation, suitable for long-term storage and having the α-galactosidase A activity for a long time, is insufficient, and a lyophilized formulation including an α-galactosidase A fusion protein at a high concentration has not been developed, and thus there is still a need to develop a lyophilized formulation including a novel composition of an α-galactosidase A fusion protein.

### Solution to Problem

An object of the present invention is to provide a lyophilized formulation including a fusion protein of α-galactosidase A.

Another object of the present invention is to provide a method for preparing the lyophilized formulation.

Another object of the present invention is to provide a method for reconstituting the lyophilized formulation.

### Advantageous Effects of Invention

The lyophilized formulation according to the present invention not only has storage stability by including a composition providing structural stability to a fusion protein of α-galactosidase A, but also has excellent stability although the fusion protein is contained at a high concentration.

### BRIEF DESCRIPTION OF DRAWINGS

FIG. 1 shows comparison results of cake appearance of a lyophilized formulation in accordance with addition of an amino acid.

FIG. 2 shows comparison results of recovery of native form of a lyophilized formulation in accordance with addition of an amino acid.

FIG. 3 shows comparison results of cake appearance of a lyophilized formulation in accordance with changes in amino acid concentration.

FIG. 4 shows comparison results of formation of soluble aggregates in a lyophilized formulation in accordance with changes in amino acid concentration before and after lyophilization.

FIG. 5 shows comparison results of recovery of native form of a lyophilized formulation in accordance with changes in amino acid concentration.

FIG. 6 is a chromatogram of a reconstituted lyophilized formulation to identify soluble aggregates (HMW1) and higher-order aggregates (HMW2) are formed in a lyophilized formulation in accordance with changes in amino acid concentration are identified.

### BEST MODE FOR CARRYING OUT THE INVENTION

An aspect of the present invention provides a lyophilized formulation including an α-galactosidase A fusion protein.

In a specific embodiment, the lyophilized formulation includes a mixture prepared by lyophilizing an aqueous solution including a fusion protein in which α-galactosidase A is linked to an immunoglobulin Fc region; a buffer; and an amino acid.

In another specific embodiment, the lyophilized formulation is characterized by including a mixture prepared by lyophilizing an aqueous solution including a fusion protein at a concentration of 5 mg/mL to 50 mg/mL; a buffer with a pH of 5.5 to 7.0; and an amino acid at a concentration of 0.5% (w/v) to 4.0% (w/v).

The lyophilized formulation according to any one of the specific embodiments is characterized by including a mixture prepared by lyophilizing an aqueous solution including a 5 mg/mL to 50 mg/mL fusion protein; a buffer with a pH of 5.5 to 7.0; 50 mM to 300 mM sodium chloride; and 0.5% (w/v) to 4.0% (w/v) serine.

The lyophilized formulation according to any one of the specific embodiments is characterized by including a mixture prepared by lyophilizing an aqueous solution including a 5 mg/mL to 50 mg/mL fusion protein; a buffer with a pH of 5.5 to 7.0; 0.002% (w/v) to 0.1% (w/v) polysorbate 20; 50 mM to 200 mM sodium chloride; and 1.0% (w/v) to 4.0% (w/v) serine.

The lyophilized formulation according to any one of the specific embodiments is characterized by including a mixture prepared by lyophilizing an aqueous solution including a 5 mg/mL to 50 mg/mL fusion protein; a buffer with a pH of 5.5 to 7.0 containing 1 mM to 30 mM histidine; 0.002% (w/v) to 0.1% (w/v) polysorbate 20; 50 mM to 200 mM sodium chloride; and 1.0% (w/v) to 4.0% (w/v) serine.

The lyophilized formulation according to any one of the specific embodiments is characterized by including a mixture prepared by lyophilizing an aqueous solution including a 5 mg/mL to 50 mg/mL fusion protein; a buffer with a pH of 5.5 to 7.0 containing 10 mM to 30 mM histidine; 0.002% (w/v) to 0.1% (w/v) polysorbate 20; 50 mM to 200 mM sodium chloride; a 0.5% (w/v) to 4.0% (w/v) sugar or sugar alcohol; and 0.5% (w/v) to 4.0% (w/v) serine.

The lyophilized formulation according to any one of the specific embodiments is characterized in that the buffer includes histidine or a salt thereof, citric acid or a salt thereof, acetic acid or a salt thereof, phosphoric acid or a salt thereof, or any combination thereof.

The lyophilized formulation according to any one of the specific embodiments is characterized in that the buffer includes 1 mM to 50 mM histidine.

The lyophilized formulation according to any one of the specific embodiments is characterized in that the amino acid is selected from the group consisting of serine, arginine, threonine, glutamine, glycine, alanine, and any combination thereof.

The lyophilized formulation according to any one of the specific embodiments is characterized in that the aqueous solution further includes a 0.5% (w/v) to 4.0% (w/v) sugar.

The lyophilized formulation according to any one of the specific embodiments is characterized in that the sugar is glucose, fructose, galactose, lactose, maltose, sucrose, trehalose, or any combination thereof.

The lyophilized formulation according to any one of the specific embodiments is characterized in that the aqueous solution further includes a 0.5% (w/v) to 4.0% (w/v) sugar alcohol.

The lyophilized formulation according to any one of the specific embodiments is characterized in that the sugar alcohol is mannitol, sorbitol, or any combination thereof.

The lyophilized formulation according to any one of the specific embodiments is characterized in that the aqueous solution further includes a 0.002% (w/v) to 0.1% (w/v) non-ionic surfactant.

The lyophilized formulation according to any one of the specific embodiments is characterized in that, the non-ionic surfactant is selected from the group consisting of poloxamer 188, polysorbate 20, polysorbate 40, polysorbate 60, polysorbate 80, and any combination thereof.

The lyophilized formulation according to any one of the specific embodiments is characterized in that the aqueous solution further includes a 5 mM to 300 mM isotonic agent.

The lyophilized formulation according to any one of the specific embodiments is characterized in that the isotonic agent is sodium chloride.

The lyophilized formulation according to any one of the specific embodiments is characterized in that the sodium chloride is contained in the aqueous solution at a concentration of 50 mM to 200 mM.

The lyophilized formulation according to any one of the specific embodiments is characterized in that the α-galactosidase A includes an amino acid sequence of SEQ ID NO: 1.

The lyophilized formulation according to any one of the specific embodiments is characterized in that the immunoglobulin Fc region includes an amino acid sequence of SEQ ID NO: 3.

The lyophilized formulation according to any one of the specific embodiments is characterized in that the α-galactosidase A fusion protein includes an amino acid sequence of SEQ ID NO: 4.

The lyophilized formulation according to any one of the specific embodiments is characterized in that the immunoglobulin Fc region is derived from IgG4.

The lyophilized formulation according to any one of the specific embodiments is characterized in that the immunoglobulin Fc region is aglycosylated.

The lyophilized formulation according to any one of the specific embodiments is characterized in that the fusion protein has a structure two molecules of α-galactosidase A are linked to each monomer of the immunoglobulin Fc region in a dimeric form.

The lyophilized formulation according to any one of the specific embodiments is characterized in that the lyophilized formulation is used to prevent or treat Fabry disease.

Another aspect of the present invention provides a method for preparing the lyophilized formulation.

In a specific embodiment, the method for preparing the lyophilized formulation is characterized by including lyophilizing an aqueous solution including an α-galactosidase A fusion protein in which α-galactosidase A is linked to an immunoglobulin Fc region, a buffer, and an amino acid.

In another specific embodiment, the method for preparing the lyophilized formulation is charactered by including further mixing the aqueous solution with an isotonic agent, a sugar, a sugar alcohol, a non-ionic surfactant, a preservative, or any combination thereof.

Another aspect of the present invention provides a method for reconstituting the lyophilized formulation including adding a reconstituting solution to the lyophilized formulation.

In a specific embodiment, the reconstituting solution is distilled water.

In another specific embodiment, a reconstituted formulation prepared by the method includes the α-galactosidase A fusion protein at a concentration of 10 mg/mL to 100 mg/mL.

### MODE FOR THE INVENTION

Hereinafter, the present invention will be described in more detail.

Meanwhile, each of the descriptions and embodiments disclosed herein may be applied herein to describe different descriptions and embodiments. That is, all of the combinations of various factors disclosed herein belong to the scope of the present invention. Furthermore, the scope of the present invention should not be limited by the detailed descriptions provided hereinbelow.

Those skilled in the art will recognize or be able to ascertain, using no more than routine experimentation, many equivalents to specific embodiments of the present invention. Such equivalents are intended to be encompassed in the scope of the following claims.

Throughout the specification, not only the conventional one-letter and three-letter codes for naturally occurring amino acids, but also those three-letter codes generally allowed for other amino acids, such as Aib (2-aminoisobutyric acid), Sar (N-methylglycine), and α-methyl-glutamic acid are used. In addition, the amino acids mentioned herein are abbreviated according to the nomenclature rules of IUPAC-IUB as follows.

| | |
|---|---|
| alanine Ala, A | arginine Arg, R |
| asparagine Asn, N | aspartic acid Asp, D |
| cysteine Cys, C | glutamic acid Glu, E |
| glutamine Gln, Q | glycine Gly, G |
| histidine His, H | isoleucine Ile, I |
| leucine Leu, L | lysine Lys, K |
| methionine Met, M | phenylalanine Phe, F |
| proline Pro, P | serine Ser, S |
| threonine Thr, T | tryptophan Trp, W |
| tyrosine Tyr, Y | valine Val, V |

An aspect of the present invention provides a lyophilized formulation including a fusion protein in which α-galactosidase A is linked to an immunoglobulin Fc region.

Specifically, the present invention relates to a lyophilized formulation including a mixture prepared by lyophilizing an aqueous solution containing an α-galactosidase A-immunoglobulin Fc region fusion protein in a pharmacologically effective amount, a buffer, and an amino acid. In addition, the aqueous solution may further include an isotonic agent, a sugar, a sugar alcohol, a non-ionic surfactant, a preservative, or any combination thereof, without being limited thereto.

As used herein, the term “lyophilized formulation” refers to a drug formulated by lyophilization. Specifically, an α-galactosidase A fusion protein may be lyophilized together with a substance used to stabilize the fusion protein such as an excipient and present in a solid state. A component contained in the lyophilized formulation together with the α-galactosidase A fusion protein, which has pharmacological efficacy of the lyophilized formulation, may be mixed with stabilizer. As used herein, the term “stabilizer” refers to a substance stably maintaining a component of a formulation such as an active ingredient for a certain period of time.

In the present invention, the lyophilized formulation is a concept including a freeze-dried substance. The lyophilized formulation is prepared by freezing a pre-formulation including both a pre-lyophilized formulation including a stabilizer for stabilizing the α-galactosidase A fusion protein and the α-galactosidase A fusion protein, and drying the resultant. In the present invention, the lyophilized formulation of the α-galactosidase A fusion protein may include a therapeutically effective amount of the α-galactosidase A fusion protein, and the therapeutically effective amount of the α-galactosidase A fusion protein may be contained in a single-use container or a multi-use container, without being limited thereto. The lyophilized formulation of the present invention has a composition capable of stabilizing the α-galactosidase A fusion protein during the lyophilization process, and even while the stored lyophilized formulation is reconstituted, stability of the formulation may be maintained. Particularly, the lyophilized formulation of the present invention has an effect on maintaining stability even when the α-galactosidase A fusion protein is contained therein at a high concentration of 5 mg/mL to 50 mg/mL.

In addition, the lyophilized formulation including the α-galactosidase A fusion protein according to the present invention in a state of being stored in a container may be reconstituted for administration to an individual.

As used herein, the term “reconstitution” refers to a process of liquifying the freeze-dried substance in a solid state such that the α-galactosidase A fusion protein is able to be administered. In this case, the concentration of the α-galactosidase A fusion protein contained in the lyophilized formulation of the present invention may be from 1 mg/mL to 150 mg/mL, from 10 mg/mL to 120 mg/mL, or from 10 mg/mL to 100 mg/mL when the lyophilized formulation is reconstituted, without being limited thereto. The concentration of the pre-formulation during a lyophilization process may be different from that after reconstitution.

The lyophilized formulation of the present invention is characterized by including a stabilizer capable of stabilizing the structure of the fusion protein such that the pharmacological efficacy of the α-galactosidase A fusion protein is retained for a long time during long-term storage. The lyophilized formulation of the present invention is characterized by including a mixture prepared by lyophilizing the aqueous solution including the α-galactosidase A fusion protein, the buffer, and the amino acid.

The α-galactosidase A fusion protein of the present invention has a structure in which α-galactosidase A is fused to an immunoglobulin Fc region. An unfolding phenomenon, in which the structure of the protein is deformed by various external factors (e.g., pH, temperature, osmosis, and presence of stabilizer), occurs during a lyophilization, reconstitution, or storage process, and causes loss of enzymatic activity resulting in a significant decrease in the pharmacological efficacy of the formulation including α-galactosidase. Particularly, one of the two domains constituting α-galactosidase A is first unfolded (to form soluble aggregates) and the other domain of α-galactosidase A and the immunoglobulin Fc region are sequentially unfolded, followed by formation of higher-order aggregates and finally, CH2 and CH3 domains of the immunoglobulin Fc region are unfolded (to form insoluble aggregates). In this case, precipitates recognizable by visual observation are formed. When the structure of the α-galactosidase A fusion protein is not maintained due to a stress generated while the lyophilized formulation is stored, soluble aggregates or higher-order aggregates are formed and the risk of immunogenicity increases, causing a safety problem.

The present inventors have found not only that including a composition inhibiting unfolding of the α-galactosidase A fusion protein is important for stability of the lyophilized formulation, but also that the enzymatic activity of α-galactosidase A is maintained since the structure of an active site is retained until insoluble aggregates are formed even after α-galactosidase A is partially unfolded and soluble aggregates are formed therefrom, and identified that it is important to add a stabilizer (e.g., amino acid) to maintain the structural stability of the α-galactosidase A fusion protein as much as possible and, furthermore, to prevent formation of insoluble aggregates caused as the unfolding phenomenon of the soluble aggregates proceeds, thereby completing the present invention. The lyophilized formulation of the present invention may have the structure and activity of the fusion protein maintained even after long-term storage and have high medication safety due to low immunogenicity.

In a specific example, the lyophilized formulation according to the present invention may include a mixture prepared by lyophilizing an aqueous solution including: a fusion protein in which α-galactosidase A is fused to an immunoglobulin Fc region; a buffer; and an amino acid, more specifically, including: a 5 mg/mL to 50 mg/mL fusion protein; a buffer with a pH of 5.5 to 7.0; and an amino acid at a concentration of 0.25% (w/v) to 4.0% (w/v).

Additionally, in another example, the lyophilized formulation may include: a 5 mg/mL to 50 mg/mL fusion protein; a buffer with a pH of 5.5 to 7.0; and an amino acid at a concentration of 0.5% (w/v) to 4.0% (w/v), wherein the pH is from 5.5 to 7.0.

Additionally, in another example, the lyophilized formulation may include a mixture prepared by lyophilizing an aqueous solution including: a 5 mg/mL to 50 mg/mL fusion protein; a buffer with a pH of 5.5 to 7.0; 50 mM to 200 mM sodium chloride; and 0.5% (w/v) to 4.0% (w/v) serine.

In another example, the lyophilized formulation may include a mixture prepared by lyophilizing an aqueous solution including: a 5 mg/mL to 50 mg/mL fusion protein; a buffer with a pH of 5.5 to 7.0; 0.002% (w/v) to 0.1% (w/v) polysorbate; 50 mM to 200 mM sodium chloride; and 0.5% (w/v) to 4.0% (w/v) serine.

In another example, the lyophilized formulation may include a mixture prepared by lyophilizing an aqueous solution including: a 5 mg/mL to 50 mg/mL fusion protein; a buffer with a pH of 5.5 to 7.0 containing 1 mM to 30 mM histidine; 0.002% (w/v) to 0.1% (w/v) polysorbate 20; 50 mM to 200 mM sodium chloride; and 1.0% (w/v) to 4.0% (w/v) serine.

In another example, the lyophilized formulation may include a mixture prepared by lyophilizing an aqueous solution including: a 5 mg/mL to 50 mg/mL fusion protein; a buffer with a pH of 5.5 to 7.0 containing 1 mM to 30 mM histidine; 0.002% (w/v) to 0.1% (w/v) polysorbate 20; 50 mM to 200 mM sodium chloride; a 0.5% (w/v) to 4.0% (w/v) sugar or sugar alcohol; and 0.5% (w/v) to 4.0% (w/v) serine, but the lyophilized formulation of the present invention is not limited to these examples.

As used herein, the term “aqueous solution” refers to a substance including the α-galactosidase A fusion protein and assisting stable storage of the α-galactosidase A fusion protein and maintaining the stability during a lyophilization process and in the case of reconstitution. Particularly, the aqueous solution includes an excipient that stabilizes the α-galactosidase A fusion protein, provides stability to the α-galactosidase A fusion protein during the lyophilization process, and allows preparation of a lyophilized formulation with storage stability. The stabilizer may include a buffer and an amino acid. Although not limited thereto, the stabilizer may further include an isotonic agent, a sugar, a sugar alcohol, a non-ionic surfactant, a preservative, and the like. In proteins such as the α-galactosidase A fusion protein, storage stability is important not only for accurate dosage but also for inhibiting potential production of an antigenic substance to the α-galactosidase A fusion protein. The aqueous solution may be used interchangeably with “pre-formulation” in the present invention.

Because the concentration of the α-galactosidase A fusion protein may be controlled by adjusting a volume of a reconstituting solution added to the lyophilized formulation, the concentration of the α-galactosidase A fusion protein in the aqueous solution is not particularly limited, but the aqueous solution may be stably lyophilized even when the α-galactosidase A fusion protein is contained at a high concentration of about 10 mg/mL or more, specifically about 10 mg/mL to 90 mg/mL, and stability of the α-galactosidase A fusion protein may be maintained in a reconstituted solution.

For example, the aqueous solution of the present invention may include the α-galactosidase A fusion protein at a concentration of about 1 mg/mL to 90 mg/mL, about 1 mg/mL to 70 mg/mL, about 1 mg/mL to 50 mg/mL, about 5 mg/mL to 50 mg/mL, about 5 mg/mL to 90 mg/mL, about 10 mg/mL to 50 mg/mL, or about 10 mg/mL to 30 mg/mL, without being limited thereto.

As used herein, the term “about” refers to a range including all of ±0.5, ±0.4, ±0.3, ±0.2, ±0.1, ±0.01, or the like and includes all numerical values equivalent to those which come immediately after the term “about” or those in a similar range, without being limited thereto.

It is generally known that as a concentration of a protein contained in a formulation increases, problems such as a decrease in stability of the formulation and an increase in precipitates occur. However, in the case where the aqueous solution including the stabilizer composition according to the present invention is lyophilized, the structure of the fusion protein during a lyophilization process and a reconstitution process without causing precipitates although the fusion protein is contained at a high concentration, and thus the aqueous solution may containing the α-galactosidase A fusion protein, as an active ingredient at a high concentration, for example, at a concentration of about 10 mg/mL or more, about 15 mg/mL or more, about 35 mg/mL or more, about 50 mg/mL or more, or about 90 mg/mL or more.

The buffer, as a component contained in the aqueous solution of the present invention, is a solution playing a role in maintaining the pH of the aqueous solution to avoid rapid change in pH of a formulation during the lyophilization process or after reconstitution. The aqueous solution of the present invention may be used as a solvent of the α-galactosidase A fusion protein and the stabilizer, and any aqueous solution may be used as the buffer of the present invention without limitation as long as the pH level in which the α-galactosidase A fusion protein is stabilized.

The pH value affects the structure of the fusion protein, and the structure of the fusion protein may be stably maintained at a pH of 5.5 to 7.0. When the pH is out of an appropriate pH range, α-galactosidase A is unfolded earlier than the immunoglobulin Fc region, resulting in impairment of structural stability. Thus, it is important to obtain structural stability of the α-galactosidase A fusion protein by maintaining the pH at an appropriate level.

The buffer may include phosphoric acid and an alkali salt thereof, as a conjugate salt (e.g., phosphate: sodium phosphate, potassium phosphate, or hydrogen or dihydrogen salt thereof), citric acid and a salt thereof (e.g., sodium citrate), acetic acid and a salt thereof (e.g., sodium acetate), or histidine and a salt thereof, and any mixture thereof may be used as the buffer, without being limited thereto. For example, the buffer may be selected from a citric acid buffer (e.g., sodium citrate buffer), an acetic acid buffer (e.g., sodium acetate buffer), a phosphoric acid buffer (e.g., sodium phosphate buffer), a histidine buffer, and any combination thereof, and the buffer or a substance used as a buffer in the aqueous solution (citric acid and a salt thereof, acetic acid and a salt thereof, histidine and a salt thereof, phosphoric acid and a salt thereof, or any combination thereof) may be contained at a concentration sufficient to maintain the pH for structural stability of proteins.

The pH of the aqueous solution may be from about 5.0 to 8.0, for example, from about 5.0 to 7.5, from about 5.0 to 7.0, from about 5.0 to 6.5, from about 5.0 to 6.0, from about 5.5 to 8.0, from about 5.5 to 7.5, from about 5.5 to 7.0, from about 5.5 to 6.5, from about 5.5 to 6.3, from about 5.5 to 6.2, from about 5.5 to 6.1, from about 5.5 to 6.0, from about 5.5 to 5.9, from about 5.5 to 5.8, from about 5.5 to 5.7, from about 5.5 to 5.6, from about 6.0 to 6.5, or from about 6.5 to 7.0, or about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0, but is not particularly limited thereto.

In order to obtain the target pH, the buffer may include phosphoric acid, citric acid, acetic acid, histidine, or salts or mixture thereof at a concentration of about 1 mM to about 200 mM, about 5 mM to about 100 mM, about 5 mM to about 80 mM, about 5 mM to about 40 mM, about 8 mM to about 40 mM, about 5 mM to about 30 mM, or about 5 mM to about 25 mM, about 10 mM to about 50 mM, about 10 mM to about 25 mM, about 15 mM to about 25 mM, or about 20 mM, without being limited thereto.

In a specific embodiment, the buffer may include about 20 mM histidine and have a pH of about 5.5 to 7.0 or about pH 5.5 to 6.5, without being limited thereto.

The amino acid contained in the aqueous solution of the present invention prevents unfolding of the α-galactosidase A fusion protein contained in the aqueous solution to inhibit formation of insoluble aggregates. Once insoluble aggregates are formed as the structural deformation (unfolding) further proceeds from the soluble aggregates, problems such as protein precipitation and loss of enzymatic activity occur.

In the Experimental Example of the present invention, it was confirmed that a lyophilized formulation including an amino acid (serine) had an appropriate appearance and a high recovery of native form, when compared with a pre-formulation not including the amino acid. Also, it was confirmed that as the concentration of the amino acid increased, specifically, when a lyophilized formulation of an aqueous solution includes an amino acid in an amount of 0.5% (w/v) or more based on a total volume of the aqueous solution, a more proper appearance was obtained and the unfolding of the α-galactosidase A fusion protein was more inhibited, thereby inhibiting formation of soluble aggregates and higher-order aggregates. In addition, the recovery of native form increased as the concentration of the amino acid increased. Because formation of the soluble aggregates or higher-order aggregates may cause immunogenicity when a formulation is administered to the body, it is required to inhibit formation of the soluble aggregates or higher-order aggregates.

The amino acid contained in the lyophilized formulation of the present invention may be serine, arginine, lysine, threonine, asparagine, glutamine, glycine, proline, alanine, valine, isoleucine, leucine, phenylalanine, or any combination thereof, specifically, may be selected from the group consisting of serine, arginine, threonine, glutamine, glycine, alanine, and any combination thereof, without being limited thereto. As a specific example, the amino acid may be serine, without being limited thereto.

Based on the total volume of the aqueous solution of the present invention, the amino acid may be present in an amount of about 0.1% (w/v) to 5.0% (w/v), about 0.25% (w/v) to 5.0% (w/v), about 0.25% (w/v) to 4.0% (w/v), about 0.5% (w/v) to 5.0% (w/v), about 0.5% (w/v) to 4.0% (w/v), about 1.0% (w/v) to 5.0% (w/v), about 1.0% (w/v) to 4.0% (w/v), about 2.0% (w/v) to 4.0% (w/v), about 2.0% (w/v) to 5.0% (w/v), about 2.5% (w/v) to 5% (w/v), about 3.0% (w/v) to 5% (w/v), about 2.0% (w/v) to 4.5% (w/v), about 2.0% (w/v) to 4.0% (w/v), or about 0.25% (w/v), 0.5% (w/v), about 1.0% (w/v), about 2.0% (w/v), about 3.0% (w/v), about 4.0% (w/v), or about 5.0% (w/v), without being limited thereto.

The aqueous solution according to the present invention may further include a non-ionic surfactant. The non-ionic surfactant refers to a substance lowering surface tension of a protein solution to prevent the protein from being adsorbed to or aggregating on a hydrophobic surface after reconstitution.

Examples of the non-ionic surfactant available in the present invention may be a polysorbate (e.g., polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate); wherein the number 20 following the polyoxyethylene indicates a total number of oxyethylene groups (—(CH2CH2O)—)), poloxamer (PEO-PPO-PEO copolymer; wherein PEO: poly(ethylene oxide) and PPO: poly(propylene oxide)), polyethylene-polypropylene glycol, a polyoxyethylene compound (e.g., polyoxyethylene-stearate, polyoxyethylene alkyl ether (alkyl: C1-C30), polyoxyethylene monoallyl ether, alkylphenyl polyoxyethylene copolymer (alkyl: C1-C30), and the like), and sodium dodecyl sulphate (SDS), or may be polysorbate or poloxamer, used alone or in a combination of at least two thereof.

Specifically, the non-ionic surfactant may be poloxamer 188, polysorbate 20, polysorbate 40, polysorbate 60, or polysorbate 80, which may be used in combination, without being limited thereto.

In the present invention, the non-ionic surfactant may be contained in the aqueous solution of the present invention at a concentration of about 0.2% (w/v) or less, for example, about 0.001% (w/v) to about 0.2% (w/v), about 0.001% (w/v) to about 0.1% (w/v), about 0.002% (w/v) to about 0.1% (w/v), about 0.01% (w/v) to about 0.1% (w/v), about 0.01% (w/v) to about 0.05% (w/v), about 0.02% (w/v) to about 0.05% (w/v), about 0.025% (w/v) to about 0.05% (w/v), or about 0.025% (w/v), based on the total volume of the aqueous solution, without being limited thereto.

The aqueous solution according to the present invention may further include an isotonic agent. The isotonic agent refers to a substance that serves to appropriately adjust an osmotic pressure in the case of administration to the body after reconstitution of the α-galactosidase A fusion protein. In addition, the isotonic agent may have an additional effect on further stabilizing the α-galactosidase A fusion protein in the aqueous solution. Any isotonic agent may be contained in the aqueous solution of the present invention without limitation as long as the isotonic agent contributes to stabilization of the α-galactosidase A fusion protein.

The isotonic agent may include a water-soluble inorganic salt, specifically, the aqueous solution of the present invention may include sodium chloride as an isotonic agent, without being limited thereto.

The concentration of sodium chloride used in the present invention may be from about 0 mM to 300 mM, specifically, about 0.1 mM to 300 mM, about 5 mM to 300 mM, about 5 mM to 200 mM, about 5 mM to 150 mM, about 5 mM to 100 mM, about 10 mM to 200 mM, about 10 mM to 150 mM, about 10 mM to 100 mM, about 30 mM to 200 mM, about 30 mM to 150 mM, about 30 mM to 100 mM, about 50 mM to 200 mM, about 50 mM to 150 mM, or about 50 mM to 100 mM, but is not limited thereto, and may be appropriately adjusted such that pre-formulations including all mixtures should be isotonic in accordance with types and amounts of components contained in the aqueous solution.

The aqueous solution according to the present invention may further include a sugar. The sugar refers to a monosaccharide, disaccharide, polysaccharide, and oligosaccharide and may increase stability of the α-galactosidase A fusion protein in the aqueous solution. Examples thereof may include mannose, glucose, fructose, galactose, fucose, lactose, maltose, sucrose, trehalose, raffinose, dextran, or any combination thereof, and in a specific embodiment, the sugar may be glucose, fructose, galactose, lactose, maltose, sucrose, trehalose, or any combination thereof, without being limited thereto. For example, the sugar may be sucrose, but is not particularly limited thereto.

The aqueous solution according to the present invention may further include a sugar alcohol, and the sugar alcohol refers to a substance including multiple hydroxyl group and includes a substance in which aldehyde groups and/or ketone groups of sugar are substituted with alcohol groups and a sugar containing multiple hydroxyl groups. The sugar or sugar alcohol may increase stability of the α-galactosidase A fusion protein. For example, the sugar alcohol may include at least one selected from the group consisting of mannitol and sorbitol, without being limited thereto.

The sugar alcohol, sugar, or any combination thereof may be present in the aqueous solution in an amount of about 0.5% (w/v) to 20% (w/v), about 0.5% (w/v) to 15% (w/v), about 0.5% (w/v) to 10% (w/v), about 0.5% (w/v) to 8% (w/v), about 0.5% (w/v) to 5% (w/v), about 0.5% (w/v) to 4.0% (w/v), about 1% (w/v) to 10% (w/v), about 1% (w/v) to 8% (w/v), about 1% (w/v) to 6% (w/v), about 1% (w/v) to 4% (w/v), or about 0.0% (w/v), about 1.0% (w/v), about 3.0% (w/v), about 4.0% (w/v), about 5.0% (w/v), or about 8.0% (w/v) based on the entire aqueous solution, without being limited thereto.

The aqueous solution according to the present invention may include different types of stabilizers in combination by adjusting the amounts thereof. For example, the aqueous solution according to the present invention may include (i) an amino acid without containing a sugar and/or sugar alcohol, or (ii) an amino acid together with a sugar and/or sugar alcohol, without being limited thereto. Although not limited thereto, in the aqueous solution including (i) an amino acid without containing a sugar and/or sugar alcohol, the content of the amino acid may be from about 1.0% (w/v) to 4.0% (w/v), and in the aqueous solution including (ii) an amino acid together with a sugar and/or sugar alcohol, the content of the sugar and/or sugar alcohol may be from about 0.5% (w/v) to 4.0% (w/v), and the content of the amino acid may be from about 0.5% (w/v) to 4.0% (w/v).

The aqueous solution according to the present invention may further include a preservative. The preservative, as a substance that substantially reduces the action of bacteria and fungi in a formulation, is added to the formulation for easy production of the formulation for multiple administration. Examples of a potential preservative may include octadecyldimethyl-benzyl ammonium chloride, hexamethonium chloride, benzalkonium chloride (a mixture of alkylbenzyldimethylammonium chloride wherein an alkyl group is a long-chain compound), and benzethonium chloride. Other types of preservative may be aromatic alcohol such as phenol, butyl, or benzyl alcohol; alkyl paraben such as methyl or propyl paraben; catechol, resorcinol, cyclohexanol, 3-pentanol, or m-cresol, without being limited thereto. The concentration of the preservative may be from 0.001% (w/v) to 1.0% (w/v), without being limited thereto.

Meanwhile, the aqueous solution (pre-formulation) for preparing the lyophilized formulation of the present invention may further optionally include other components or substances known in the art in addition to the above-described buffer, isotonic agent, amino acid, non-ionic surfactant, sugar alcohol, sugar, and preservative within a range that does not impair the effect of the present invention, without being limited thereto.

As a specific example, the aqueous solution (pre-formulation) for preparing the lyophilized formulation according to the present invention may be an aqueous solution including a 5 mg/mL to 50 mg/mL fusion protein; a buffer with a pH of 5.5 to 7.0; and 0.5% (w/v) to 4.0% (w/v) serine, without being limited thereto.

As another specific example, the aqueous solution (pre-formulation) for preparing the lyophilized formulation according to the present invention may be an aqueous solution including a 5 mg/mL to 50 mg/mL fusion protein; a buffer with a pH of 5.5 to 7.0; a 0.002% (w/v) to 0.1% (w/v) non-ionic surfactant; and 0.5% (w/v) to 4.0% (w/v) serine, without being limited thereto.

As another specific example, the aqueous solution (pre-formulation) for preparing the lyophilized formulation according to the present invention may be an aqueous solution including a 5 mg/mL to 50 mg/mL fusion protein; a buffer with a pH of 5.5 to 7.0 containing 10 mM to 30 mM histidine; 0.002% (w/v) to 0.1% (w/v) polysorbate; and 1.0% (w/v) to 4.0% (w/v) serine, without being limited thereto.

As another specific example, the aqueous solution (pre-formulation) for preparing the lyophilized formulation according to the present invention may be an aqueous solution including a 5 mg/mL to 50 mg/mL fusion protein; a buffer with a pH of 5.5 to 7.0; 0.002% (w/v) to 0.1% (w/v) polysorbate 20; 50 mM to 200 mM sodium chloride; and 1.0% (w/v) to 4.0% (w/v) serine, without being limited thereto.

As another specific example, the aqueous solution (pre-formulation) for preparing the lyophilized formulation according to the present invention may be an aqueous solution including a 5 mg/mL to 50 mg/mL fusion protein; 10 mM to 30 mM histidine; 0.002% (w/v) to 0.1% (w/v) polysorbate 20; 50 mM to 200 mM sodium chloride; a 0.5% (w/v) to 4.0% (w/v) sugar or sugar alcohol; and 0.5% (w/v) to 4.0% (w/v) serine, without being limited thereto.

In addition, the aqueous solution for preparing the lyophilized formulation or the lyophilized formulation may further include a pharmaceutically acceptable carrier, an excipient, and the like, and this carrier or excipient may be non-naturally occurring.

The aqueous solution for preparing the lyophilized formulation of the present invention may be frozen and dried under proper freezing and drying conditions well known in the art. The drying may be completed by a primary process or a secondary or more processes.

In addition, the lyophilized formulation of the present invention may be prepared by lyophilizing a pre-formulation including an appropriate concentration of the α-galactosidase A fusion protein in consideration of a desire dosage, and reconstituting the resultant to be suitable for administration to an individual. In addition, the pre-formulation may be diluted to increase the volume and lyophilized, and then reconstituted by diluting with a reconstituting solution in a smaller volume than that of the lyophilized formulation, without being limited thereto. In the present invention, the lyophilized formulation may be a mixture prepared by lyophilizing an aqueous solution including the α-galactosidase A fusion protein and a stabilizer (e.g., buffer and amino acid), and also be a reconstituted formulation prepared by reconstituting the lyophilized formulation using a reconstituting solution, without being limited thereto.

Meanwhile, hereinafter, the α-galactosidase A fusion protein contained in the lyophilized formulation of the present invention, as an active ingredient, will be described in more detail.

The α-galactosidase A fusion protein contained in the lyophilized formulation of the present invention refers to a fusion protein in which α-galactosidase A is linked to an immunoglobulin Fc region, and the fusion protein may have a structure in which two molecules of α-galactosidase A are linked to the dimeric immunoglobulin Fc region via a linker. Specifically, two α-galactosidase A molecules may be linked by covalent bonds to each monomer of the immunoglobulin Fc region in a dimeric form via a linker. In addition, the two α-galactosidase A molecules may form a dimer via a non-covalent bond, but is not limited thereto. The fusion protein of the present invention has increased stability of α-galactosidase A since the immunoglobulin Fc region is fused to the α-galactosidase A and thus the pharmacological efficacy may be maintained for a long time in the body. The lyophilized formulation of the present invention does not lose the pharmacological efficacy even after long-term storage by providing excellent stability to the fusion protein by lyophilizing a pre-formulation containing the fusion protein and a stabilizer.

Specifically, the fusion protein of the present invention may have an amino acid sequence of SEQ ID NO: 4, or may be encoded by a polynucleotide having a nucleotide sequence of SEQ ID NO: 5, without being limited thereto. The fusion protein of the present invention may be in a form in which two monomers including the amino acid sequence of SEQ ID NO: 4 form a dimer, without being limited thereto. For the fusion protein of the present invention, the specification of WO 2019/009684 is incorporated as a reference.

As used herein, the terms “fusion protein in which α-galactosidase A is linked to an immunoglobulin Fc region are fused”, “α-galactosidase A fusion protein”, and “fusion protein” may be interchangeably used.

The fusion protein of the present invention is expressed in a transformant in a form where α-galactosidase A is linked to the immunoglobulin Fc region via a linker such that α-galactosidase A forms a dimer via a non-covalent bond when the immunoglobulin Fc region forms a dimer.

The α-galactosidase A (α-Gal A, GLA) of the present invention, which is an enzyme present in lysosomes of spleen, brain, liver, and the like and hydrolyzes α-galactosyl moieties at ends of glycolipids and glycoproteins, is a homodimeric glycoprotein. Particularly, α-galactosidase A is known to be associated with Fabry disease that is a lysosomal storage disease. The α-galactosidase A has a dimeric structure consisting of two domains (TIM barrel domain and β-sheet containing immunoglobulin-like domain) (Journal of Biological chemistry, Vol. 287, No. 34, 2012; Lieberman et al. Biochemistry, Vol. 48, No. 22, 2009), and because an unfolding phenomenon occurs more easily as the pH increases, the pH of a formulation containing α-galactosidase A is required to be appropriately adjusted. Particularly, while the active site is maintained in the state of soluble aggregates formed as one domain is unfolded, the activity is lost once insoluble aggregates are formed as the unfolding further proceeds, the formulation is required to include a stabilizer to keep the enzyme in the active state by maintaining the soluble aggregate state. Furthermore, since the soluble aggregates increase the risk of immunogenicity, there is a high risk of inducing immune response when the soluble aggregates are administered to the body. Therefore, it is important to increase medication safety by increasing stability of a formulation to prevent formation of soluble aggregates as much as possible during storage of the formulation.

In the present invention, the α-galactosidase A may be a native form or a recombinant form, specifically, may include an amino acid sequence of SEQ ID NO: 1, without being limited thereto.

In addition, the α-galactosidase A of the present invention includes fragments of the native form or analogs thereof in which one or several amino acids are altered by one selected from the group consisting of substitution, addition, deletion, modification, and any combination thereof, without limitation, as long as they have activity identical or equivalent to that of the native form of the enzyme.

Additionally, the α-galactosidase A may include an amino acid sequence having at least 60%, 70%, or 80%, specifically at least 90%, more specifically, at least 91%, 92%, 93%, 94%, 95%, 96%, 97%, 98%, or 99% homology with the amino acid sequence of SEQ ID NO: 1 and may be obtained from microorganisms by using a genetic engineering method or be commercially available, without being limited thereto.

As used herein, the term “homology” refers to a degree of identity with a wild-type amino acid sequence or a wild-type nucleotide sequence, and the homology comparison may be done by visual observation or using a commercially available comparison program. Using a commercially available computer program, the homology between two or more sequences may be expressed as a percentage (%). The homology (%) may be calculated between adjacent sequences.

Genetic information of sequences of the α-galactosidase A or derivatives thereof and nucleotide sequences encoding the same may be available from known database such as the National Center for Biotechnology Information (NCBI).

The α-galactosidase A fusion protein of the present invention may be prepared by expressing the fusion protein in a transformant in a form where α-galactosidase A is linked to the immunoglobulin Fc region via a linker.

The linker is a peptide linker, and the fusion protein of the present invention may be in a form in which the α-galactosidase A is linked to the immunoglobulin Fc region via the peptide linker. One end of the linker may be linked to one of the chains of the immunoglobulin Fc region in a dimer form, without being limited thereto.

The peptide linker may include one or more amino acids, e.g., 1 to 1000 amino acids, and include any peptide linker known in the art, such as [GS]x linker, [GGGS]x linker, and [GGGGS]x linker, wherein x is a natural number of 1 or greater (e.g., 1, 2, 3, 4, 5, or more), without being limited thereto. Specifically, the peptide linker of the present invention may consist of 10 to 50 amino acids, more specifically, 20 to 40 amino acids and may have an amino acid sequence of SEQ ID NO: 2.

In view of the objects of the present invention, sites of the α-galactosidase A and the immunoglobulin Fc region to which the peptide linker is linked are not particularly limited as long as the activity of the α-galactosidase A is retained after being linked to the immunoglobulin Fc region. Specifically, the sites may be both termini of the α-galactosidase A and the immunoglobulin Fc region, more specifically, the C-terminus of the α-galactosidase A and the N-terminus of the immunoglobulin Fc region, without being limited thereto.

As used herein, the term “N-terminus” or “C-terminus” refers to the amino terminus and the carboxyl terminus of the respective proteins. Examples thereof include, but are not limited to, not only the last amino acid residues of the N-terminus or the C-terminus but also amino acid residues near the N-terminus or the C-terminus, specifically, up to the 10^{th} amino acid residues from the last amino acid.

In the present invention, the peptide linkers may be linked to each of the monomers of the immunoglobulin Fc region in a dimeric form, and the linkers respectively linked to the immunoglobulin Fc region monomers of the dimer may be independently linked to the α-galactosidase A, without being limited thereto.

The immunoglobulin Fc region, one of the moieties constituting the enzyme fusion protein of the present invention, may be a dimer formed of immunoglobulin Fc region monomers.

As used herein, the term “immunoglobulin Fc region” refers to a region including a heavy chain constant domain 2 (CH2) and/or a heavy chain constant domain 3 (CH3) excluding the heavy chain and light chain variable domains of immunoglobulin. In view of the objects of the present invention, the immunoglobulin Fc region may include a modified hinge region, without being limited thereto. Specifically, the immunoglobulin Fc region may be one in which one or more amino acids are altered from the native form of the immunoglobulin Fc region by one selected from the group consisting of substitution, addition, deletion, modification, and any combination thereof, without being limited thereto.

The immunoglobulin Fc region is a substance used as a carrier in drug production. In order to stabilize a protein and prevent the protein from being eliminated by the kidney, extensive research has been conducted into fusion proteins using the immunoglobulin Fc region in recent years. Immunoglobulins are major constituents of the blood, and there are five different types, i.e., IgG, IgM, IgA, IgD, and IgE. The most frequently used type for fusion protein studies is IgG, and it is classified into four subtypes (IgG1 to IgG4).

The immunoglobulin Fc region may include a hinge region in the heavy chain constant domain, the immunoglobulin Fc region monomers may constitute a dimer via the hinge region, without being limited thereto. In addition, the immunoglobulin Fc region of the present invention may be an extended Fc region including a part of or the entirety of a heavy chain constant domain 1 (CH1) and/or a light chain constant domain 1 (CL1) excluding the heavy chain and the light chain variable domains of the immunoglobulin, as long as the immunoglobulin Fc region has substantially identical or enhanced effects compared to the native form. In addition, the immunoglobulin Fc region may be a region from which a considerably long part of the amino acid sequence corresponding to the CH2 and/or CH3 is eliminated.

Specifically, the immunoglobulin Fc region of the present invention may include 1) a CH1 domain, a CH2 domain, a CH3 domain, and a CH4 domain, 2) a CH1 domain and a CH2 domain, 3) a CH1 domain and a CH3 domain, 4) a CH2 domain and a CH3 domain, or 5) a combination of one or more domains selected from a CH1 domain, a CH2 domain, a CH3 domain, and a CH4 domain and an immunoglobulin hinge region (or a part of the hinge region), but is not limited thereto. More specifically, the immunoglobulin Fc region may include a hinge region, a CH2 domain, and a CH3 domain, but is not limited thereto.

As used herein, the term “hinge sequence” refers to a site located at a heavy chain and forming the dimer of the immunoglobulin Fc region via an inter disulfate bond. In the present invention, the dimer is formed of two molecules of the immunoglobulin Fc chain via the hinge sequence of the immunoglobulin Fc region.

Specifically, the hinge region may be one where a part of the hinge region is deleted to have only one cysteine (Cys) residue, or one where a serine (Ser) residue involved in chain exchange is substituted with a proline (Pro) residue. More specifically, the hinge region may be one where the 2^{nd} serine residue is substituted with a proline residue, without being limited thereto. The immunoglobulin Fc region of the present invention may have an amino acid sequence of SEQ ID NO: 3, without being limited thereto.

The immunoglobulin Fc region of the present invention include not only a native sequence obtained from papain digestion of immunoglobulin but also derivatives, substitution products, and variants thereof, e.g., sequences different from the native sequence and obtained by modification of one or more amino acid residues by deletion, addition, non-conservative or conservative substitution, or a combination thereof. The derivatives, substitution products, and variants are those having the ability to binding FcRn.

For example, in the case of IgG Fc, amino acid residues known to be important in linkage at positions 214 to 238, 297 to 299, 318 to 322, or 327 to 331 may be used as a suitable site for modification.

Additionally, other various types of derivatives including those in which a site capable of forming a disulfide bond is deleted or certain amino acid residues are eliminated from the N-terminus of a native Fc form, and a methionine residue is added to the N-terminus of the native Fc form may be used. In addition, to remove effector functions, a complement-binding site, such as a C1q-binding site, may be deleted, and an antibody dependent cell mediated cytotoxicity (ADCC) site may be deleted. Techniques of preparing such sequence derivatives of the immunoglobulin Fc region are disclosed in International Patent Publication Nos. WO 97/34631 and WO 96/32478.

Amino acid exchanges in proteins and peptides, which do not generally alter the activity of molecules, are known in the art (H. Neurath, R. L. Hill, The Proteins, Academic Press, New York, 1979). The most commonly occurring exchanges of amino acid residues are exchanges between Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. If required, the Fc region may be modified by phosphorylation, sulfation, acrylation, glycosylation, methylation, farnesylation, acetylation, and amidation.

The above-described sequence derivatives of the immunoglobulin Fc region are derivatives that have biological activity equivalent to the immunoglobulin Fc region of the present invention and improved structural stability against heat, pH, or the like.

In addition, these immunoglobulin Fc regions may be obtained from native forms isolated from humans and other animals including cows, goats, swine, mice, rabbits, hamsters, rats and guinea pigs, or may be recombinants or derivatives thereof, obtained from transformed animal cells or microorganisms. In this regard, they may be obtained from a native immunoglobulin by isolating whole immunoglobulins from living humans or animals and treating them with a protease. Papain digests the native immunoglobulin into Fab and Fc regions and pepsin digests the native immunoglobulin into pF′c and F(ab)2 fragments. These fragments may be subjected to size-exclusion chromatography to isolate Fc or pF′c. In a more specific embodiment, a human-derived immunoglobulin Fc region is a recombinant immunoglobulin Fc region obtained from a microorganism.

In addition, the immunoglobulin Fc region may have natural glycans or increased or decreased glycans compared to the natural type, or be in a deglycosylated form. The increase, decrease, or removal of glycans of the immunoglobulin Fc may be achieved by any methods commonly used in the art such as a chemical method, an enzymatic method, and a genetic engineering method using a microorganism. In this regard, the immunoglobulin Fc region obtained by removing glycans shows a significant decrease in binding affinity to a complement c1q and a decrease in or loss of antibody-dependent cytotoxicity or complement-dependent cytotoxicity, and thus unnecessary immune responses are not induced thereby in living organisms. Based thereon, a deglycosylated or aglycosylated immunoglobulin Fc region may be more suitable as a drug carrier for its own purpose.

As used herein, the term “deglycosylation” refers to a Fc region from which glycan is removed using an enzyme and the term “aglycosylation” refers to a Fc region that is not glycosylated and produced in prokaryotes, more specifically, E. coli.

Meanwhile, the immunoglobulin Fc region may be derived from humans or animals such as cows, goats, swine, mice, rabbits, hamsters, rats, or guinea pigs. In a more specific embodiment, the immunoglobulin Fc region may be derived from humans.

In addition, the immunoglobulin Fc region may be derived from IgG, IgA, IgD, IgE, or IgM, or any combination or hybrid thereof. In a more specific embodiment, the immunoglobulin Fc region is derived from IgG or IgM which are the most abundant proteins in human blood, and in an even more specific embodiment, it is derived from IgG known to enhance the half-lives of ligand-binding proteins. In a yet even more specific embodiment, the immunoglobulin Fc region is an IgG4 Fc region, and in the most specific embodiment, the immunoglobulin Fc region is an aglycosylated Fc region derived from human IgG4, without being limited thereto. The immunoglobulin Fc region of the present invention may have an amino acid sequence of SEQ ID NO: 3, but is not limited thereto. More specifically, the immunoglobulin Fc region may include a monomer having an amino acid sequence of SEQ ID NO: 3, and the immunoglobulin Fc region may be a homodimer of the monomers having an amino acid sequence of SEQ ID NO: 3, without being limited thereto.

Meanwhile, as used herein, the term “combination” refers to formation of a linkage between a polypeptide encoding a single-chain immunoglobulin Fc region of the same origin and a single-chain polypeptide of a different origin when a dimer or a multimer is formed. That is, a dimer or multimer may be prepared using two or more Fc fragments selected from the group consisting of IgG Fc, IgA Fc, IgM Fc, IgD Fc, and IgE Fc fragments.

The immunoglobulin Fc region of the α-galactosidase A fusion protein may be in a dimeric form, specifically, may have a structure in which two polypeptide chains are linked to each other via a disulfide bond. More specifically, the two chains may be linked via the nitrogen atom of one of the two chains, without being limited thereto. The linkage via the nitrogen atom may be formed via reductive amination of an ε-amino group of lysine or an N-terminal amino group, without being limited thereto. In a specific embodiment, the immunoglobulin Fc region may be linked to the linker by a nitrogen atom of proline at the N-terminus, without being limited thereto. One Fc region of the dimeric form may be linked to two α-galactosidase A molecules by covalent bonds via two linkers, without being limited thereto.

Unless otherwise stated, detailed descriptions about the α-galactosidase A or fusion protein of the present invention disclosed in the specification or the claims may be applied not only to the α-galactosidase A or fusion protein but also to a salt thereof (e.g., pharmaceutically acceptable salt), or a solvate form thereof. Thus, although only “α-galactosidase A” or “fusion protein” are described in the specification, descriptions thereof may also be applied to particular salts thereof, particular solvates thereof, and solvates of the particular salts. Such salts may be, for example, in the form of a pharmaceutically acceptable salt. Types of the salts are not particularly limited. However, the salts may be in a form safe and effective in mammals, without being limited thereto.

The term “pharmaceutically acceptable” refers to a substance that may be effectively used for the intended use within the scope of pharmaco-medical decision without inducing excessive toxicity, irritation, allergic responses, and the like.

As used herein, the term “pharmaceutically acceptable salt” refers to a salt derived from a pharmaceutically acceptable inorganic acid, organic acid, or base. Examples of a suitable acid may include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, naphthalene-2-sulfonic acid, and benzenesulfonic acid. Examples of the salt derived from a suitable base may include alkali metals such as sodium and potassium, alkali earth metals such as magnesium, and ammonium.

In addition, as used herein, the term “solvate” refers to a complex of the α-galactosidase A, fusion protein or the salt thereof according to the present invention and a solvent molecule.

The fusion protein may be prepared or produced by any method known in the art, specifically, may be obtained from animal cells into which an expression vector is inserted after culturing and purifying the animal cells, or may be synthesized based on the sequence thereof, without being limited thereto.

The lyophilized formulation of the present invention may be used to prevent, treat, and alleviate a disease, such as α-galactosidase A deficiency, whose symptoms may be prevented, treated, or alleviated by administering the α-galactosidase A fusion protein, without being limited thereto.

The α-galactosidase A fusion protein of the present invention may be used as a drug of an enzyme replacement therapy (ERT). The enzyme replacement therapy may prevent or treat a disease by recovering hypofunction of an enzyme by supplementing the deficient or insufficient enzyme that causes the disease.

Specifically, the lyophilized formulation of the present invention may be used to prevent, treat, or alleviate α-galactosidase A deficiency. The α-galactosidase A deficiency is a lysosomal storage disease caused by deficiency of α-galactosidase A (α-Gal A) that is a lysosomal enzyme and include Fabry disease, Angiokeratoma Diffuse, Angiokeratoma Corporis Diffusum, or Hereditary Dystopic Lipidosis.

Fabry disease, one of the lysosomal storage diseases, is a recessively inherited disorder caused by X-chromosomal inactivation. Fabry disease is a congenital metabolic disorder of glycolipid (glycosphingolipid) caused by deficient or insufficient activity of α-galactosidase A. It has been known that abnormal accumulation of globotriaosylceramide (Gb3) on the blood vessel wall and various parts of the body, such as skin, kidneys, heart, and nervous system, caused by the abnormality of α-galactosidase A, significantly affects blood circulation and reduces supply of nutrients. Symptoms such as hypohidrosis, acroparesthesia, severe pain, angiokeratoma, corneal opacity, cardiac ischemia, myocardial infarction, and renal failure are caused, and eventually the kidneys failed to function properly, leading to death.

As used herein, the term “prevention” refers to all actions that inhibit or delay a disease by administering the fusion protein or the lyophilized formulation including the same. As used herein, the term “treatment” refers to all actions that ameliorate or beneficially change symptoms of a target disease by administering the fusion protein or the lyophilized formulation including the same.

In a specific embodiment of the present invention, the lyophilized formulation of the present invention may be used to prevent or treat the Fabry disease, without being limited thereto.

In addition, the lyophilized formulation of the present invention may be administered via a subcutaneous route, but is not limited thereto. Specifically, the lyophilized formulation according to the present invention may be reconstructed to be suitable for subcutaneous administration, but is not limited thereto.

As used herein, the term “administration” refers to introduction of the reconstituted lyophilized formulation into a patient by any suitable method, and administration routes of the lyophilized formulation are not particularly limited, but the lyophilized formulation may be administered by any general route as long as the α-galactosidase A fusion protein of the lyophilized formulation reaches a target in the living body, for example, intraperitoneal administration, intravenous administration, intramuscular administration, subcutaneous administrations, intradermal administration, oral administration, topical administration, intranasal administration, intrapulmonary administration, or rectal administration may be used, without being limited thereto.

A preferred daily dosage of the fusion protein of the present invention may be from about 0.0001 mg to 500 mg per 1 kg of the body weight of a patient and the lyophilized formulation of the present invention may be formulated such that the daily dosage is properly administered or reconstructed such that the daily dosage is administered. However, the dosage of the fusion protein is determined as an effective dosage for the patient in consideration of various factors such as age, body weight, health status, and gender of the patient, severity of disease, diet, and excretion rate as well as administration route and number of administration thereof, and thus an appropriate effective dosage for a particular use of the lyophilized formulation of the present invention may be determined by one of ordinary skill in the art in consideration of these factors.

Another aspect of the present invention provides an aqueous solution or a pre-lyophilized formulation including an α-galactosidase A fusion protein, a buffer, and an amino acid for preparing the lyophilized formulation.

The aqueous solution or pre-lyophilized formulation may further include at least one selected from an isotonic agent, a sugar or sugar alcohol, and a non-ionic surfactant, without being limited thereto.

The aqueous solution, the pre-lyophilized formulation, the lyophilized formulation, and components constituting the same are as described above.

Another aspect of the present invention provides a method for preparing the lyophilized formulation.

Specifically, the preparation method may include lyophilizing an aqueous solution including an α-galactosidase A fusion protein, a buffer, and an amino acid.

The aqueous solution or lyophilized formulation may further include at least one selected from an isotonic agent, a sugar or sugar alcohol, and a non-ionic surfactant, without being limited thereto.

The aqueous solution, the lyophilized formulation, and components constituting the same are as described above.

Another aspect of the present invention provides a method for reconstituting the lyophilized formulation, the method including adding a reconstituting solution to the lyophilized formulation.

The aqueous solution, the lyophilized formulation, and reconstitution are as described above.

As used herein, the term “reconstituting solution” refers to a solution used to reconstitute the lyophilized formulation by adding the reconstituting solution to the lyophilized formulation in a solid state. Examples of the reconstituting solution may include distilled water (water), without being limited thereto.

The reconstituted formulation may include the α-galactosidase A fusion protein at a concentration suitable for administration to an individual, specifically, may include the α-galactosidase A fusion protein in an amount of 10 mg/mL to 100 mg/mL, 10 mg/mL to 90 mg/mL, 10 mg/mL to 50 mg/mL, or 10 mg/mL to 30 mg/mL, without being limited thereto.

The reconstituted formulation may be obtained by reconstituting the lyophilized formulation of the present invention to be suitable for subcutaneous administration to an individual, without being limited thereto.

Hereinafter, the present invention will be described in more detail with reference to the following examples and experimental examples. However, the following examples and experimental examples are merely presented to exemplify the present invention, and the scope of the present invention is not limited thereto.

### Example 1: Preparation of Fusion Protein of α-Qalactosidase a and Immunoglobulin Fc Region

A fusion protein of α-galactosidase A and an immunoglobulin Fc region (hereinafter referred to as α-galactosidase A fusion protein, SEQ ID NO: 4), in which native-type α-galactosidase A (SEQ ID NO: 1) is linked to an immunoglobulin Fc region (SEQ ID NO: 3) via a linker (SEQ ID NO: 2), was prepared.

Specifically, a polynucleotide (SEQ ID NO: 5) encoding the α-galactosidase A fusion protein was inserted into an XOGC vector, which is an expression vector, by using a restriction enzyme to prepare a vector that expresses the ax-galactosidase A fusion protein.

DNA and protein sequences of the α-galactosidase A fusion protein are as shown in Table 1 below. In the protein sequence of Table 1 below, underlines indicate signal sequences, bold letters indicate amino acid substitution, and italic letters indicate the linker. The α-galactosidase A fusion protein of the present invention is in a dimeric form consisting of two monomers having the amino acid sequence of SEQ ID NO: 4.

TABLE 1

| | | SEQ ID |
|---|---|---|
| | Sequence | NO: |
| Protein | MQLRNPELHL GCALALRFLA LVSWDIPGAR | 4 |
| | ALDNGLARTP TMGWLHWERF MCNLDCQEEP | |
| | DSCISEKLFM EMAELMVSEG WKDAGYEYLC | |
| | IDDCWMAPQR DSEGRLQADP QRFPHGIRQL | |
| | ANYVHSKGLK LGIYADVGNK TCAGFPGSFG | |
| | YYDIDAQTFA DWGVDLLKFD GCYCDSLENL | |
| | ADGYKHMSLA LNRTGRSIVY SCEWPLYMWP | |
| | FQKPNYTEIR QYCNHWRNFA DIDDSWKSIK | |
| | SILDWTSFNQ ERIVDVAGPG GWNDPDMLVI | |
| | GNFGLSWNQQ VTQMALWAIM AAPLFMSNDL | |
| | RHISPQAKAL LQDKDVIAIN QDPLGKQGYQ | |
| | LRQGDNFEVW ERPLSGLAWA VAMINRQEIG | |
| | GPRSYTIAVA SLGKGVACNP ACFITQLLPV | |
| | KRKLGFYEWT SRLRSHINPT GTVLLQLENT | |
| | MQMSLKDLLG GGGSGGGGSG GGGSGGGGSG | |
| | GGGSGGGGSP PCPAPEFLGG PSVFLFPPKP | |
| | KDTLMISRTP EVTCVVVDVS QEDPEVQFNW | |
| | YVDGVEVHNA KTKPREEQFQ STYRVVSVLT | |
| | VLHQDWLNGK EYKCKVSNKG LPSSIEKTIS | |
| | KAKGQPREPQ VYTLPPSQEE MTKNQVSLTC | |
| | LVKGFYPSDI AVEWESNGQP ENNYKTTPPV | |
| | LDSDGSFFLY SRLTVDKSRW QEGNVFSCSV | |
| | MHEALHNHYT QKSLSLSLGK | |
| DNA | ATGCAGCTGA GGAACCCAGA ACTACATCTG | 5 |
| | GGCTGCGCGC TTGCGCTTCG CTTCCTGGCC | |
| | CTCGTTTCCT GGGACATCCC TGGGGCTAGA | |
| | GCACTGGACA ATGGATTGGC AAGGACGCCT | |
| | ACCATGGGCT GGCTGCACTG GGAGCGCTTC | |
| | ATGTGCAACC TTGACTGCCA GGAAGAGCCA | |
| | GATTCCTGCA TCAGTGAGAA GCTCTTCATG | |
| | GAGATGGCAG AGCTCATGGT CTCAGAAGGC | |
| | TGGAAGGATG CAGGTTATGA GTACCTCTGC | |
| | ATTGATGACT GTTGGATGGC TCCCCAAAGA | |
| | GATTCAGAAG GCAGACTTCA GGCAGACCCT | |
| | CAGCGCTTTC CTCATGGGAT TCGCCAGCTA | |
| | GCTAATTATG TTCACAGCAA AGGACTGAAG | |
| | CTAGGGATTT ATGCAGATGT TGGAAATAAA | |
| | ACCTGCGCAG GCTTCCCTGG GAGTTTTGGA | |
| | TACTACGACA TTGATGCCCA GACCTTTGCT | |
| | GACTGGGGAG TAGATCTGCT AAAATTTGAT | |
| | GGTTGTTACT GTGACAGTTT GGAAAATTTG | |
| | GCAGATGGTT ATAAGCACAT GTCCTTGGCC | |
| | CTGAATAGGA CTGGCAGAAG CATTGTGTAC | |
| | TCCTGTGAGT GGCCTCTTTA TATGTGGCCC | |
| | TTTCAAAAGC CCAATTATAC AGAAATCCGA | |
| | CAGTACTGCA ATCACTGGCG AAATTTTGCT | |
| | GACATTGATG ATTCCTGGAA AAGTATAAAG | |
| | AGTATCTTGG ACTGGACATC TTTTAACCAG | |
| | GAGAGAATTG TTGATGTTGC TGGACCAGGG | |
| | GGTTGGAATG ACCCAGATAT GTTAGTGATT | |
| | GGCAACTTTG GCCTCAGCTG GAATCAGCAA | |
| | GTAACTCAGA TGGCCCTCTG GGCTATCATG | |
| | GCTGCTCCTT TATTCATGTC TAATGACCTC | |
| | CGACACATCA GCCCTCAAGC CAAAGCTCTC | |
| | CTTCAGGATA AGGACGTAAT TGCCATCAAT | |
| | CAGGACCCCT TGGGCAAGCA AGGGTACCAG | |
| | CTTAGACAGG GAGACAACTT TGAAGTGTGG | |
| | GAACGACCTC TCTCAGGCTT AGCCTGGGCT | |
| | GTAGCTATGA TAAACCGGCA GGAGATTGGT | |
| | GGACCTCGCT CTTATACCAT CGCAGTTGCT | |
| | TCCCTGGGTA AAGGAGTGGC CTGTAATCCT | |
| | GCCTGCTTCA TCACACAGCT CCTCCCTGTG | |
| | AAAAGGAAGC TAGGGTTCTA TGAATGGACT | |
| | TCAAGGTTAA GAAGTCACAT AAATCCCACA | |
| | GGCACTGTTT TGCTTCAGCT AGAAAATACA | |
| | ATGCAGATGT CATTAAAAGA CTTACTTGGC | |
| | GGCGGAGGTT CAGGTGGTGG TGGCTCTGGC | |
| | GGTGGAGGGT CGGGGGGAGG CGGCTCTGGA | |
| | GGAGGGGGCT CCGGTGGGGG AGGTAGCCCA | |
| | CCATGCCCAG CACCTGAGTT CCTGGGGGGA | |
| | CCATCAGTCT TCCTGTTCCC CCCAAAACCC | |
| | AAGGACACCC TCATGATCTC CCGGACCCCT | |
| | GAGGTCACAT GCGTGGTGGT GGACGTGAGC | |
| | CAGGAAGACC CTGAGGTCCA GTTCAACTGG | |
| | TACGTGGACG GCGTGGAGGT GCATAATGCC | |
| | AAGACAAAGC CGCGGGAGGA GCAGTTCCAA | |
| | AGCACGTACC GTGTGGTCAG CGTCCTCACC | |
| | GTCCTGCACC AGGACTGGCT GAATGGCAAG | |
| | GAGTACAAGT GCAAGGTCTC CAACAAAGGC | |
| | CTCCCATCCT CCATCGAGAA AACCATCTCC | |
| | AAAGCCAAAG GGCAGCCCCG AGAACCACAG | |
| | GTGTACACCC TGCCCCCATC CCAGGAGGAG | |
| | ATGACCAAGA ACCAGGTCAG CCTGACCTGC | |
| | CTGGTCAAAG GCTTCTATCC CAGCGACATC | |
| | GCCGTGGAGT GGGAGAGCAA TGGGCAGCCG | |
| | GAGAACAACT ACAAGACCAC GCCTCCCGTG | |
| | CTGGACTCCG ACGGCTCCTT CTTCCTCTAC | |
| | AGCAGGCTAA CCGTGGACAA GAGCAGGTGG | |
| | CAGGAGGGGA ACGTCTTCTC ATGCTCCGTG | |
| | ATGCATGAGG CTCTGCACAA CCACTACACG | |
| | CAGAAGAGCC TCTCCCTGTC TCTGGGTAAA | |
| | TGA | |

CHO-S cell lines were transfected with a vector (pX0GC-alpha galactosidase-Fc) that expresses the α-galactosidase A fusion protein prepared as described above to prepare cells lines capable of mass producing the α-galactosidase A fusion protein.

Specifically, CHO-S cells were suspension-cultured in a 1 L Erlenmeyer flask (Corning, cat. No. 431147) using a serum-free culture medium (FreeStyle CHO Expression Medium, Thermo Fisher, cat. No. 12651014) until the number of cells in the culture vessel reached 5×10⁸, and then the cells were transformed using a FreeStyle Max (Thermo Fisher, cat. No. 16447-100). That is, after adding 10 mL of OptiPro SFM (Thermo Fisher, cat. No. 12309-019) to each of the two tubes, and 500 μg of DNA was added to one tube and 500 μL of the FreeStyle Max was added to the other tube, followed by mixing the two solutions, and the mixture was allowed to stand at room temperature for 10 minutes. Then, the culture medium was replaced with a new FreeStyle CHO expression medium (Thermo Fisher, cat. No. 12651014). The cells were incubated under the conditions of 37° C., 5% C02, 125 rpm for about 96 hours to prepare α-galactosidase A fusion protein.

In the α-galactosidase A fusion protein prepared as described above, each of the two immunoglobulin Fc region monomers constituting the dimer is linked to α-galactosidase A to form a structure in which the immunoglobulin Fc region in the dimeric form is fused to two α-galactosidase A molecules.

### Example 2: Identification of Stability of Lyophilized Formulation According to Addition of Amino Acid

### Example 2-1: Preparation and Reconstitution of Lyophilized Formulation

With respect to the lyophilized formulation including the α-galactosidase A fusion protein prepared in Example 1, in order to identify changes in stability of the lyophilized formulation according to addition of an amino acid, various lyophilized formulations were prepared and appearance analysis and size-exclusion chromatography were conducted on the lyophilized formulations.

For the preparation of the lyophilized formulations, each of the solutions of the formulations having the compositions shown in Table 2 below were aliquoted into a glass vial (3 ml) by 1.0 ml, half-turned with a Rubber stopper, and loaded on a shelf of a freeze dryer (Lyostar 3, SP scientific). Subsequently, lyophilization was conducted under the conditions shown in Table 3 below, and the prepared lyophilized formulation was capped with an aluminum cap after completion of aluminum lyophilization.

TABLE 2

| | | | | | Amino acid |
|---|---|---|---|---|---|
| | Concentration | | | | (L-Serine |
| # | of fusion protein | Buffer | Isotonic agent | pH | (%, (w/v))) |
| 1 | 10 mg/mL | 20 mM L-histidine | 50 mM sodium | 6.0 | — |
| | | | chloride | | |
| 2 | 10 mg/mL | 20 mM L-histidine | 150 mM sodium | 6.0 | — |
| | | | chloride | | |
| 3 | 10 mg/mL | 20 mM L-histidine | 200 mM sodium | 6.0 | — |
| | | | chloride | | |
| 4 | 10 mg/mL | 20 mM L-histidine | 300 mM sodium | 6.0 | — |
| | | | chloride | | |
| 5 | 10 mg/mL | 20 mM L-histidine | — | 6.0 | 2.0 |

TABLE 3

| | | | Primary | Secondary |
|---|---|---|---|---|
| | Loading | Freezing | drying | drying |
| Temperature (° C.) | 4.0 | −60 | −25 | 20 |
| Heating rate (° C./min) | 0 | 1.0 | 1.0 | 1.0 |
| Holding time (min) | 30 | 300 | 2000 | 1200 |
| Pressure (mTorr) | N/A | N/A | 40 | 40 |

Stability of the prepared lyophilized formulation was identified SE-HPLC analysis after reconstitution using 1.0 ml of distilled water (DW).

### Example 2-2: Size Exclusion Liquid Chromatography (SE-HPLC)

For size-exclusion chromatography, first, the reconstituted lyophilized formulation was diluted using a mobile phase (1×PBS, Lonza) to a concentration of 1.0 mg/mL, followed by sterile filtration, and 200 μL of the filtered sample was injected into a vial insert and prepared in a screw top vial.

Subsequently, after connecting the mobile phase to a pump, an assay column (TSKgel G3000SWXL, Tosoh) was loaded on Waters e2695 and Waters 2489 devices (manufactured by Waters, Japan) while flowing the mobile phase at a flow rate of 0.5 mL/min. The sample was equilibrated by flowing the mobile phase at a flow rate of 0.5 mL/min for 30 minutes or more until detector signals were stabilized. When the temperature of an autosampler was lowered to 4° C., the sample was inserted into the sampler. After injecting 10 μL of the sample, detection peaks were identified at 214 nm while flowing the mobile phase for 35 minutes. The results were analyzed using Empower Pro software of a personal computer (PC).

### Example 2-3: Identification of Stability of Lyophilized Formulation According to Addition of Amino Acid

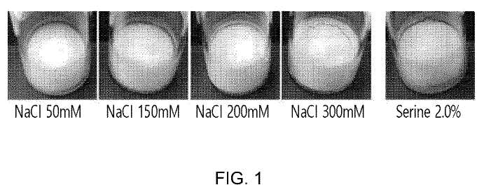

First, as a result of comparison between cake appearances of the lyophilized formulations prepared in Example 2-1, although partial collapse was observed in the formulation including a high-concentration isotonic agent (NaCl), serious collapse was not observed in the lyophilized formulation, and suitable appearances were observed. In the case of reconstitution, the lyophilized formulation to which serine was added exhibited a transparent appearance indicating excellent appearance (FIG. 1).

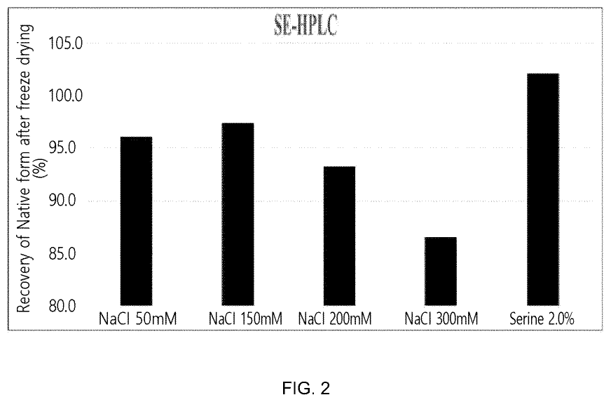

In addition, as a result of identifying the recovery of native form from the prepared lyophilized formulation by size-exclusion chromatography, it was confirmed that the rate of recovery of native form was 100% or less in the formulations to which serine was not added and the recovery of native form decreased as the concentration of the isotonic agent (NaCl) increased, but the recovery of native form was 102.0% in the formulation containing serine indicating that the formulation was stable (FIG. 2).

In this regard, the native form, as the α-galactosidase A fusion protein prepared in Example 1, was a fusion protein in an intact dimeric form in which aggregation or degradation did not occur. The definition may be applied to the following examples in the same manner.

Comparison results of stability of lyophilized formulations to which an amino acid (serine) was added are shown in Table 4 below.

TABLE 4

| | | | Recovery of |
|---|---|---|---|
| # | Cake appearance | Appearance after dissolving | native form % |
| 1 | elegant | slightly turbid, no bubbles | 96.0 |
| 2 | elegant | turbid, no bubbles | 97.4 |
| 3 | elegant | turbid, no bubbles | 93.2 |
| 4 | partially collapsed | turbid, no bubbles | 86.5 |
| 5 | elegant | transparent, no bubbles, excellent | 102.0 |
| | | appearance after dissolving | |

### Example 3: Identification of Stability of Lyophilized Formulation According to Amino Acid Concentration

### Example 3-1: Preparation and Reconstitution of Lyophilized Formulation

First, in order to identify changes in stability of the lyophilized formulation according to the concentration of the amino acid in the lyophilized formulation containing the α-galactosidase A fusion protein prepared in Example 1, various lyophilized formulations were prepared and appearance analysis and size-exclusion chromatography were conducted on the lyophilized formulations.

Each of the formulations having the compositions shown in Table 5 below was aliquoted into a glass vial (3 ml) by 0.5 ml, half-turned with a Rubber stopper, and loaded on a shelf of a freeze dryer (Lyostar 3, SP scientific). Subsequently, lyophilization was conducted under the conditions shown in Table 6 below, and the prepared lyophilized formulation was capped with an aluminum cap after completion of aluminum lyophilization.

TABLE 5

| | Concentration | | | | Amino acid |
|---|---|---|---|---|---|
| | of fusion | | | Non-ionic | (L-Serine |
| # | protein | Buffer | pH | surfactant | (%, (w/v))) |
| 1 | 30 mg/mL | 10 mM L-histidine | 5.5 | 0.025% | 0.25 |
| | | | | polysorbate 20 | |
| 2 | 30 mg/mL | 10 mM L-histidine | 5.5 | 0.025% | 0.5 |
| | | | | polysorbate 20 | |
| 3 | 30 mg/mL | 10 mM L-histidine | 5.5 | 0.025% | 1.0 |
| | | | | polysorbate 20 | |
| 4 | 30 mg/mL | 10 mM L-histidine | 5.5 | 0.025% | 2.0 |
| | | | | polysorbate 20 | |
| 5 | 30 mg/mL | 10 mM L-histidine | 5.5 | 0.025% | 4.0 |
| | | | | polysorbate 20 | |

TABLE 6

| | | | | Secondary |
|---|---|---|---|---|
| | Loading | Freezing | Primary drying | drying |
| Temperature (° C.) | 4.0 | −60 | −25 | 20 |
| Heating rate (° C./min) | 0 | 1.0 | 1.0 | 1.0 |
| Holding time (min) | 30 | 300 | 2000 | 1200 |
| Pressure (mTorr) | N/A | N/A | 40 | 40 |

Stability of the prepared lyophilized formulation was identified SE-HPLC analysis after reconstitution using 0.25 ml of distilled water (DW). The compositions of the reconstituted lyophilized formulations are as shown in Table 7 below, and SE-HPLC analysis was performed in the same manner as in Example 2-2.

TABLE 7

| | Concentration | | | | Amino acid |
|---|---|---|---|---|---|
| | of fusion | | | Non-ionic | (L-Serine |
| # | protein | Buffer | pH | surfactant | (%, (w/v))) |
| 1 | 60 mg/mL | 20 mM L-histidine | 5.5 | 0.05% | 0.5 |
| | | | | polysorbate 20 | |
| 2 | 60 mg/mL | 20 mM L-histidine | 5.5 | 0.05% | 1.0 |
| | | | | polysorbate 20 | |
| 3 | 60 mg/mL | 20 mM L-histidine | 5.5 | 0.05% | 2.0 |
| | | | | polysorbate 20 | |
| 4 | 60 mg/mL | 20 mM L-histidine | 5.5 | 0.05% | 4.0 |
| | | | | polysorbate 20 | |
| 5 | 60 mg/mL | 20 mM L-histidine | 5.5 | 0.05% | 8.0 |
| | | | | polysorbate 20 | |

### Example 3-2: Identification of Stability of Lyophilized Formulation According to Amino Acid Concentration

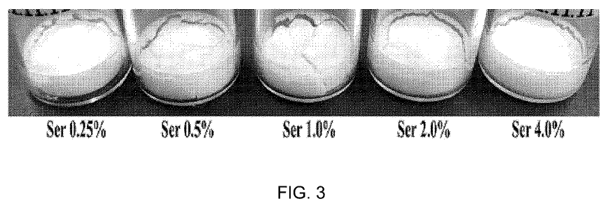

As a result of comparing cake appearance of the lyophilized formulations according to the concentration of the amino acid, serious collapse was not observed in all of the lyophilized formulations and appropriate appearance were observed (FIG. 3).

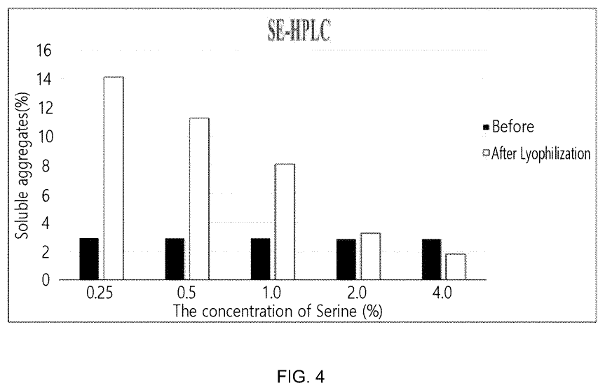

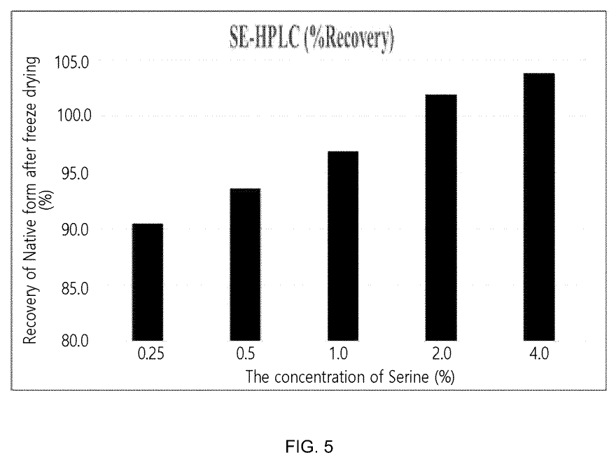

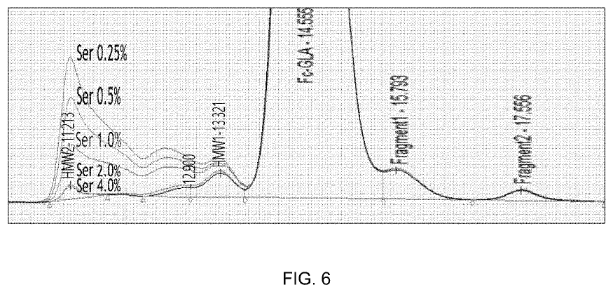

In addition, based on size-exclusion chromatography, it was confirmed that as the concentration of serine increased in the lyophilized formulation, formation of soluble aggregates decreased (FIG. 4), the recovery of native form increased (FIG. 5), and the progress from soluble aggregates (HMW1) to higher-order aggregates (HMW2) was more inhibited (FIG. 6). Particularly, in the lyophilized formulation containing serine at a concentration of 2.0% or more, it was confirmed that the recovery of native form was greater than 100% when compared with that before lyophilization.

In this regard, the soluble aggregates formed as some domains of the α-galactosidase A are unfolded have the activity of α-galactosidase A while having solubility, and the higher-order aggregates are aggregates formed as the unfolding of the soluble aggregates proceeds. The native form refers to the α-galactosidase A fusion protein, prepared in Example 1, and is a fusion protein in an intact dimeric form that has not undergone aggregation or degradation. As higher-order aggregates are formed and the size of aggregates increases, the risk of immunogenicity increases, and thus safety of the lyophilized formulation may be increased by inhibiting formation of the higher-order aggregates by adding serine.

These experimental results indicate that the lyophilized formulation containing the amino acid has high safety, and particularly, as the concentration of the amino acid increases, formation of aggregates is more inhibited, thereby more easily maintaining purity of the α-galactosidase A fusion protein.

Comparison results of stability of the lyophilized formulation containing the amino acid (serine) confirmed as described above are listed in Table 8 below.

TABLE 8

| | | | Recovery of |
|---|---|---|---|
| # | Cake appearance | Appearance after dissolving | native form (%) |
| 1 | partially collapsed | clear and transparent, no bubbles | 90.4 |
| 2 | partially collapsed | clear and transparent, no bubbles | 93.5 |
| 3 | partially collapsed | clear and transparent, no bubbles | 96.9 |
| 4 | partially collapsed | clear and transparent, no bubbles | 101.9 |
| 5 | partially collapsed | clear and transparent, no bubbles | 103.8 |

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention. Furthermore, the scope of the present invention should be defined by the appended claims rather than the detailed description, and it should be understood that all modifications or variations derived from the meanings and scope of the present invention and equivalents thereof are included in the scope of the present invention.

1. A lyophilized formulation comprising a fusion protein in which α-galactosidase A is linked to an immunoglobulin Fc region, the lyophilized formulation comprising a mixture prepared by lyophilizing aqueous solution comprising:
the fusion protein at a concentration of 5 mg/mL to 50 mg/mL;
a buffer with a pH of 5.5 to 7.0; and
an amino acid at a concentration of 0.5% (w/v) to 4.0% (w/v).

2. The lyophilized formulation according to claim 1, wherein the buffer comprises histidine or a salt thereof, citric acid or a salt thereof, acetic acid or a salt thereof, phosphoric acid or a salt thereof, or any combination thereof; or wherein the buffer comprises 1 mM to 50 mM histidine.

3. (canceled)

4. The lyophilized formulation according to claim 1, wherein the amino acid is selected from the group consisting of serine, arginine, threonine, glutamine, glycine, alanine, and any combination thereof.

5. The lyophilized formulation according to claim 1, wherein the aqueous solution further comprises a sugar at a concentration of 0.5% (w/v) to 4.0% (w/v).

6. The lyophilized formulation according to claim 5, wherein the sugar is glucose, fructose, galactose, lactose, maltose, sucrose, trehalose, or any combination thereof.

7. The lyophilized formulation according to claim 1, wherein the aqueous solution further comprises a sugar alcohol at a concentration of 0.5% (w/v) to 4.0% (w/v).

8. The lyophilized formulation according to claim 7, wherein the sugar alcohol is mannitol, sorbitol, or any combination thereof.

9. The lyophilized formulation according to claim 1, wherein the aqueous solution further comprises a non-ionic surfactant at a concentration of 0.002% (w/v) to 0.1% (w/v).

10. The lyophilized formulation according to claim 9, wherein the non-ionic surfactant is selected from the group consisting of poloxamer 188, polysorbate 20, polysorbate 40, polysorbate 60, polysorbate 80, and any combination thereof.

11. The lyophilized formulation according to claim 1, wherein the aqueous solution further comprises an isotonic agent at a concentration of 5 mM to 300 mM.

12. The lyophilized formulation according to claim 11, wherein the isotonic agent is sodium chloride.

13. The lyophilized formulation according to claim 1, wherein the α-galactosidase A comprises an amino acid sequence of SEQ ID NO: 1; or wherein the fusion protein comprises an amino acid sequence of SEQ ID NO: 4.

14. (canceled)

15. The lyophilized formulation according to claim 1, wherein the fusion protein has a structure in which two molecules of α-galactosidase A are linked to each monomer of an immunoglobulin Fc region in a dimeric form.

16. The lyophilized formulation according to claim 1, wherein the lyophilized formulation comprises a mixture prepared by lyophilizing an aqueous solution comprising:
the fusion protein at a concentration of 5 mg/mL to 50 mg/mL;
10 mM to 30 mM histidine;
0.002% (w/v) to 0.1% (w/v) polysorbate 20;
50 mM to 200 mM sodium chloride; and
1.0% (w/v) to 4.0% (w/v) serine.

17. The lyophilized formulation according to claim 1, wherein the lyophilized formulation comprises a mixture prepared by lyophilizing an aqueous solution comprising:
the fusion protein at a concentration of 5 mg/mL to 50 mg/mL;
10 mM to 30 mM histidine;
0.002% (w/v) to 0.1% (w/v) polysorbate 20;
50 mM to 200 mM sodium chloride;
a sugar or sugar alcohol at a concentration of 0.5% (w/v) to 4.0% (w/v); and
0.5% (w/v) to 4.0% (w/v) serine.

18. The lyophilized formulation according to claim 1, wherein the lyophilized formulation is used to prevent or treat Fabry disease.

19. A method for preparing the lyophilized formulation according to claim 1, the method comprising lyophilizing an aqueous solution comprising an α-galactosidase A fusion protein in which α-galactosidase A is linked to an immunoglobulin Fc region, a buffer, and an amino acid.

20. A method for reconstituting the lyophilized formulation according to claim 1, the method comprising adding a reconstituting solution to the lyophilized formulation according to claim 1.

21. The method according to claim 20, wherein the reconstituting solution is distilled water.

22. The method according to claim 20, wherein a reconstituted formulation prepared by the method comprises the α-galactosidase A fusion protein at a concentration of 10 mg/mL to 100 mg/mL.
